(19) United States
(12) Patent Application Publication
(10) Pub. No.: US 2025/0275682 A1
(43) Pub. Date: Sep. 4, 2025

(54) INFRARED RADIAL ARTERY VISUALIZATION DEVICE

(71) Applicant: Logistics and Supply Chain MultiTech R&D Centre Limited, Pok Fu Lam (HK)

(72) Inventors: Leung Chiu, Pok Fu Lam (HK); Kin Keung Lee, Pok Fu Lam (HK); Ka Lun Fan, Pok Fu Lam (HK)

(21) Appl. No.: 18/592,924

(22) Filed: Mar. 1, 2024

(51) Int. Cl. A61B 5/02; A61B 5/024

(52) U.S. Cl. CPC A61B 5/02007; A61B 5/02433

(57) ABSTRACT

An infrared radial artery visualization device includes an infrared light source; an infrared light sensor positioned opposite the infrared light source; and a wrist support adapted to hold a wrist between the infrared light source and the sensor wherein the sensor is adapted to capture infrared light emitted from the infrared light source and penetrated through the wrist

### FIELD OF THE INVENTION

The present invention relates to a device for showing radial artery using infrared light. More particularly, the present invention relates to a device for facilitating the identification of acupoints by employing infrared light to highlight the radial artery.

### BACKGROUND OF THE INVENTION

The diagnostic approach within the framework of Traditional Chinese Medicine (TCM) emphasizes the significance of observation, auditory perception, inquiry, and palpation. This methodology places a considerable reliance on interpersonal interaction between practitioners and patients.

The pulse diagnosis in traditional Chinese medicine is one of the most central diagnostic technique in the four diagnostic methods of TCM and is also the most commonly used and exploratory diagnostic means. The pulse-taking of traditional Chinese medicine is also called “pulse-feeling” and involves the use fingers of the doctors or practitioners to press cun, guan, and chi (qi) acupoints of the cun-kou radial artery of patients so as to assess the state of an illness and distinguish the condition or disease of the patient by sensing the pulse-beating condition.

Since the COVID-19 pandemic, tele-practice has been heavily promoted. However, Traditional Chinese Medicines (TCM) tele-practice is not fully implemented because palpation is required in many TCM applications and is difficult via stand-alone video conferencing technology.

Conventionally, Chinese Medicine Practitioners (CMP) feels pressure changes by fingers in a face-to-face consultation. Without instrumental analysis, the practitioner practicing TCM, by using their expertise, can use their fingers by applying varying pressures on the patient's arteries to read pulses based on their own experiences and sensations. In turn, the CMP can then express the reading by words to form a diagnosis. However, this diagnostic step cannot be carried out remotely and thus remote practice of TCM may be difficult without this diagnostic step.

### SUMMARY OF THE INVENTION

In accordance with a first aspect of the present invention, there is provided an Infrared Radial Artery Visualization Device comprising: an infrared light source; an infrared light sensor positioned opposite the infrared light source; and a wrist support adapted to hold a wrist between the infrared light source and the sensor wherein the sensor is adapted to capture infrared light emitted from the infrared light source and penetrated through the wrist to generate infrared wrist image or video.

In accordance with the first aspect, the infrared light source is adapted to generate light with a wavelength from around 750 nm to 1000 μm (400 THz to 300 GHz).

In accordance with the first aspect, the infrared light source is adapted to emit infrared light in the near-infrared region of the electromagnetic spectrum (from 780 nm to 2500 nm).

In accordance with the first aspect, the infrared light source comprises one or more light-emitting diodes (LED).

In accordance with the first aspect, the LEDs may be arranged in an array or a panel format.

In accordance with the first aspect, the infrared light source comprises a circuit for modulating the cycle, pulse width, intensity, and wavelength of the infrared light emission.

In accordance with the first aspect, the infrared light source may comprise a temperate regulation system for regulating the temperature of the infrared light source.

In accordance with the first aspect, the temperature regulation system may comprise one or more vents and/or fans.

In accordance with the first aspect, the temperature regulation system may comprise a temperature sensor.

In accordance with the first aspect, the light source 22 is secured in an enclosure with an opening.

In accordance with the first aspect, the opening comprises a transparent plate or a semi-opaque shading to diffuse the infrared light.

In accordance with the first aspect, the enclosure comprises a prism, a reflector, and/or lens to direct or focus the infrared light.

In accordance with the first aspect, the infrared light sensor is connected to a frame.

In accordance with the first aspect, the frame forms an arch with a substantially rectangular shape.

In accordance with the first aspect, the infrared light sensor may be connected to the frame at a hinge such that the infrared light sensor may be pivotable and/or rotatable relative to the frame.

In accordance with the first aspect, the frame is secured to the platform.

In accordance with the first aspect, the platform comprises fringes to assist in positing the wrist over the infrared light sensor.

In accordance with the first aspect, the fringes portion 16 may be molded as an integral part of the platform.

In accordance with the first aspect, the infrared sensor comprises one or more Charge Coupled Device (CCD) and Complementary Metal-Oxide-Semiconductor (CMOS) sensors.

In accordance with the first aspect, the infrared sensor is adapted to communicate with a processor for processing image signals through one or more communication interfaces.

In accordance with the first aspect, a diffusion lens is configured on top of the infrared light source.

In accordance with the first aspect, Infrared Radial Artery Visualization Device comprises a data communication module to transmit the infrared wrist image or video for presenting on two or more display interfaces.

### BRIEF DESCRIPTION OF THE DRAWINGS

Embodiments of the present invention will now be described, by way of example, with reference to the accompanying drawings in which:

FIG. 1 is a schematic diagram of a radial artery viewing device according to an embodiment of the present invention;

FIG. 2 is a schematic diagram of the radial artery in the wrist region with an infrared source and infrared camera of the radial artery viewing device of FIG. 1;

FIG. 3 is a schematic diagram of the acupoints of palpation on the radial artery in the wrist region of FIG. 2;

FIG. 4 is an exemplified image of a subject's left wrist captured by the radial artery viewing device of FIG. 1;

FIG. 5 is an exemplified image of a subject's right wrist captured by the radial artery viewing device of FIG. 1;

FIG. 6 is an exemplified image of the acupoints of palpation recognition on the infrared image generated by the radial artery viewing device of FIG. 1;

FIG. 7 is a schematic diagram of the radial artery viewing device displaying images on different locations; and

FIG. 8 is a schematic diagram of another embodiment of the radial artery in the wrist region with an infrared source and infrared camera of the radial artery viewing device of FIG. 1.

### DETAILED DESCRIPTION OF ILLUSTRATIVE EMBODIMENTS

Palpation is one of the four diagnostic methods in traditional Chinese medicine (TCM) and it is very important for Chinese medicine practitioners to perform this diagnosis on their patients. Palpation involves the systematic assessment of the alterations in the body's pulse. The pulse is induced by the relaxation and contraction of the aortic wall in response to cardiac rhythm. This vascular phenomenon is further characterized by a wave-like fluctuation at the artery, commonly referred to as the pulse wave, detectable around the wrist. This cardiac rhythm may cause changes in blood flow volume and pressure in arteries manifested through different pulse patterns. These pulse patterns, in accordance with TCM studies, may be observed at the cun, guan, and chi (qi) acupoints which happen to coincide with the radial artery, as it is known in Western medicine.

The pulse patterns reading at the cun, guan, and chi acupoints used to be carried out in person by a Chinese Medicine Practitioner or TCM doctor. In order to assist with the practice of tele-medicine or the tele-Traditional Chinese Medicine, embodiments of the present invention provide an Infrared Radial Artery Visualization Device 10 to fill the gap in TCM tele-practice and new technologies for both Chinese medicine practitioners and patients by allowing for the palpation to be taken remotely. Suitable positions are critical in TCM palpation and thus an example of an Infrared Radial Artery Visualization device 10 is arranged to indicate the suitable acupoints.

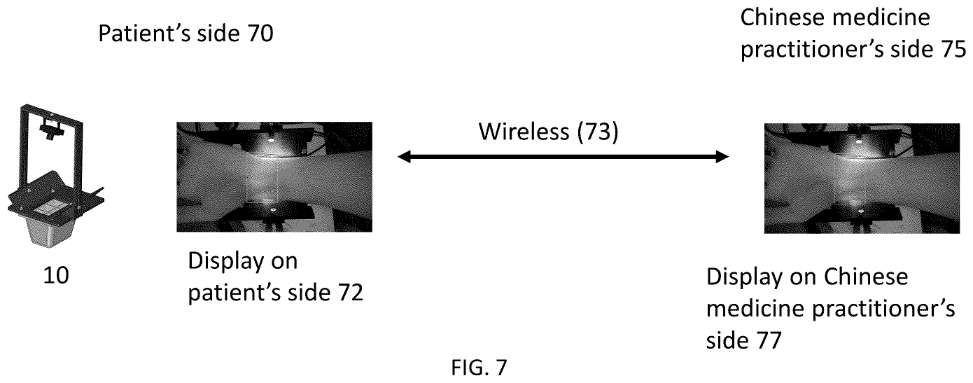

In TCM tele-practice, Chinese medicine practitioners and patients may be located in two different places away from each other. Palpation may be achieved using a remote system with the Infrared Radial Artery Visualization Device 10 as shown in FIG. 7.

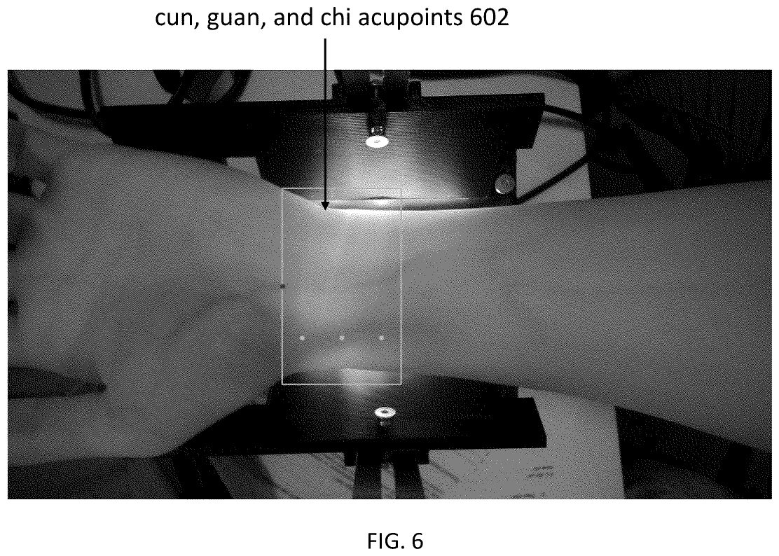

In this embodiment of the present invention, the Infrared Radial Artery Visualization Device 10 is located at the patient's side 70. The Infrared Radial Artery Visualization Device 10 is associated with a display interface 72 to present the infrared wrist image/video on the patient's side. The display interface may be an LCD, LED, or OLED display or monitor connected to the Infrared Radial Artery Visualization Device 10. In another embodiment, the Infrared Radial Artery Visualization Device 10 is in communication with a computer system or smart device (not shown) on the patient's side 70 and is adapted to display the infrared wrist image/video as captured by the Device 10 so as to show the visualization of the artery. Further information on this display is shown, in one example, in an enlarged format FIG. 6, which shows that the display 72 and 77 would allow the visualization of cun, guan and chi acupoints 602 on the infra-red wrist image for the patient and practitioner.

In one embodiment, Infrared Radial Artery Visualization Device 10 comprises a data communication module to transmit (73) the infrared wrist image or video for presenting on two or more display interfaces. The Infrared Radial Artery Visualization Device 10 is adapted to send the infrared wrist image or video to be displayed at the Chinese medicine practitioner's side 75, which may also have a display 77. In one embodiment, the data communication module is adapted to transmit data via a wireless (73) network such as Bluetooth™, WIFI, or cellar network. In one embodiment, the Infrared Radial Artery Visualization Device 10 has an Internet of Things (IoT) form factor.

In one embodiment of the present invention, the infrared wrist image/video is transferred to the Chinese Medicine Practitioner by wireless network. Both the infrared wrist image/video is displayed on both Chinese medicine practitioner's and patient's sides. The recognition of cun, guan, and chi acupoints will provide suggestions to assist the Chinese Medicine Practitioner in finding the required acupoints, but it does not replace the work of the Chinese Medicine Practitioner. The Chinese Medicine Practitioner reads the image/video displayed on a monitor and is able to instruct patients through video conference software or remotely adjust the device to suitable positions.

In another embodiment of the present invention, the real-time presentation of the infrared wrist image or video can be displayed on the displays at the Chinese medicine practitioner's side and the patient's side. In one embodiment, the system situated at the Chinese medicine practitioner's side is adapted to receive input for generating overlaying information on the infrared wrist image or video. This overlay is simultaneously projected onto the displays at both the Chinese medicine practitioner's and the patient's interfaces in real-time.

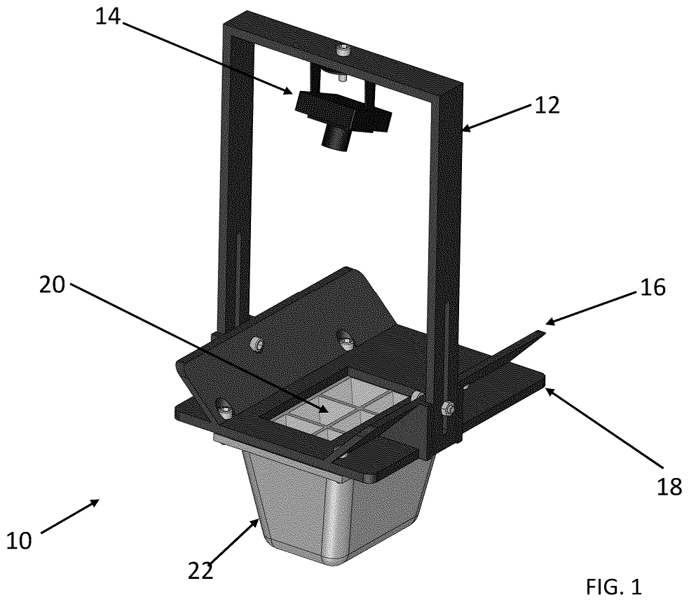

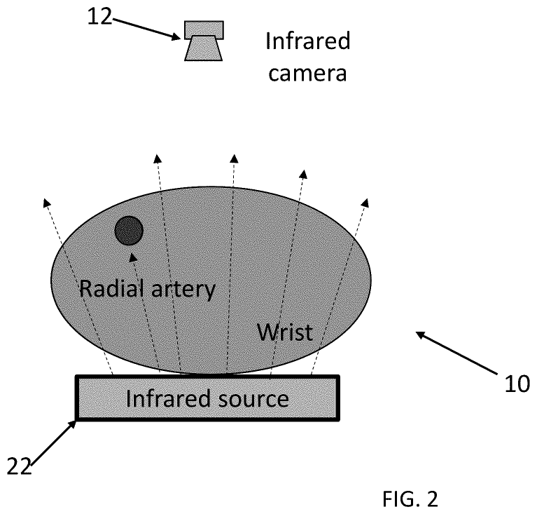

With reference to FIG. 1 and FIG. 2, there is illustrated an Infrared Radial Artery Visualization device 10 in accordance with one embodiment of the present invention. In this embodiment, the Infrared Radial Artery Visualization device 10 comprises an infrared light source 22, an infrared light sensor 12 positioned opposite the infrared light source, and a platform 18 adapted to hold or support a wrist between the infrared light source and the sensor such that the sensor is adapted to capture infrared light emitted from the infrared light source that had penetrated through the wrist to generate infrared wrist image or video.

In one embodiment, the infrared light source 12 is adapted to generate light with a wavelength of around 750 nm to 1000 μm (300 GHz to 400 THz). In one embodiment, the infrared light source is adapted to emit infrared light in the near-infrared region of the electromagnetic spectrum (from 780 nm to 2500 nm). In one embodiment, the infrared light source 12 comprises one or more light-emitting diodes (LED) adapted to emit infrared light. The LEDs may be arranged in an array or a panel format. In one embodiment, the infrared light source 22 comprises a circuit for modulating the cycle, pulse width, intensity, and wavelength of the infrared light emission. In another embodiment, the infrared light source 22 may comprise a temperate regulation system for regulating the temperature of the infrared light source. The temperature regulation system may comprise one or more vents and/or fans to allow airflow through the infrared light source 22. In yet another embodiment, the temperature regulation system may comprise a temperature sensor to measure the temperature of the infrared light source 22.

The infrared light source is not a single source but comprises multiple different LEDs or light sources emitting different wavelengths. The wavelength of infrared light can be selected by turning on different LEDs or light sources. Different wavelengths have varying levels of penetration through the wrist and different absorption levels. A sharper image can be selected by different wavelength or created by using different wavelengths than when using a single wavelength.

In this embodiment, the light source 22 is secured in an enclosure with an opening 20. The opening 20 may comprise a transparent plate. In another embodiment, the enclosure may have a prism, a reflector, and/or lens to direct or focus the infrared light. In another embodiment, the opening 20 may comprise a semi-opaque shading to diffuse the infrared light.

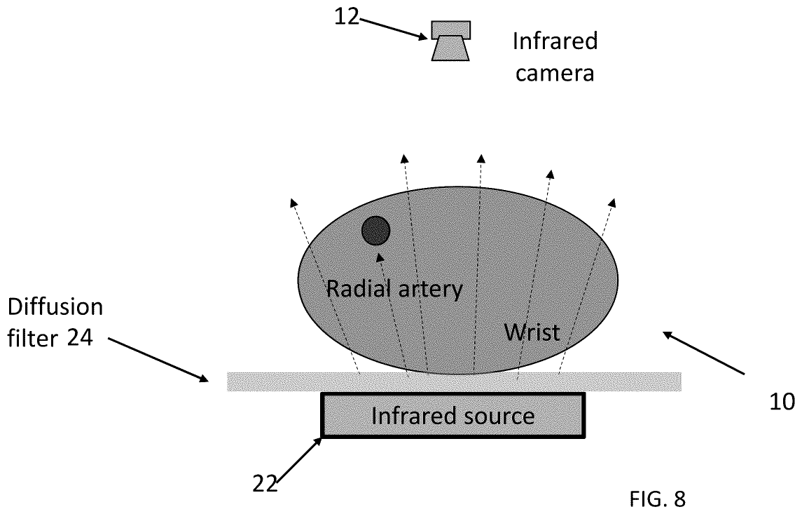

In accordance with one embodiment as shown in FIG. 8, a diffusion lens 24 is configured on top of the infrared light source.22 of the Infrared Radial Artery Visualization Device 10. The infrared light source 22 is covered by a diffusion lens 24. The function of the diffusion lens 24 is to provide a relatively uniform infrared intensity, improving the image and resulting in a clearer distribution of blood vessels. Both arteries and veins can be displayed on the monitor.

In one embodiment of the present invention, as shown in FIG. 1, the infrared light sensor 12 is connected to a frame 14. In one embodiment, the frame 14 forms an arch with a substantially rectangular shape. In another embodiment, the frame 14 form an arch of a smooth curve. The infrared light sensor 12 may be connected to the frame 14 at a hinge such that the infrared light sensor may be pivotable and/or rotatable relative to the frame. In another embodiment, the infrared light sensor 12 is adapted to slide along the frame 14. In one embodiment of the present invention, the frame 14 is secured to the platform 18. The frame 14 may be rotatable about the platform 18 such that the frame and the platform may stay on the same plane. It is also envisaged in one embodiment that the frame 14 may slide along the edge of the platform 18. Preferably, the height of the frame 14 may be adjustable.

Referring to FIG. 1, which illustrates an embodiment of the present invention, the platform 18 may comprise flanges 16 to assist in positing the wrist over the infrared light sensor 12. The flanges 16 may be molded as an integral part of the platform 18.

Preferably, the infrared sensor 12 comprises one or more Charge Coupled Device (CCD) and Complementary Metal-Oxide-Semiconductor (CMOS) sensors. The infrared sensor 12 may comprise a reflector to focus the infrared light. The infrared sensor 12 may also comprise a wide-angle lens to cover the angle of view between 60° and 100°. The infrared sensor 12 may comprise one or more optical filters to block noises. The optical filter may be one or more of a long-pass filter, short-pass filter, and polarizing filter.

In one embodiment, infrared sensor 12 is adapted to communicate with a processor for processing image signals through one or more communication interfaces. In one embodiment, the communication interface comprises a Universal Serial Bus (USB) circuit for connecting to a processor directly. In another embodiment, the communication interface comprises a wireless communication circuit for transferring image data captured by the infrared sensor 12 to a remote processor. The Infrared Radial Artery Visualization Device 10 may also comprise data storage for storing images data captured by the infrared sensor 12 internally.

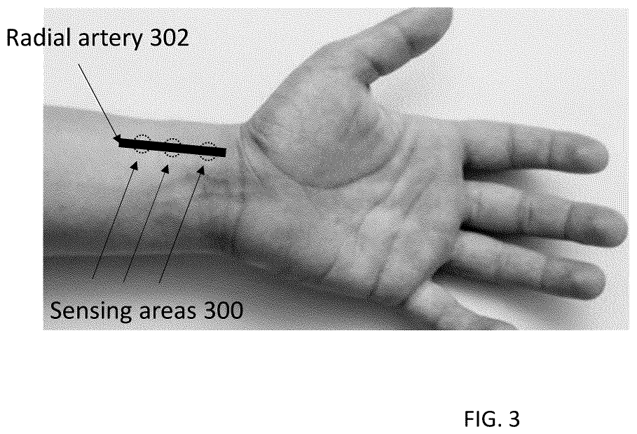

The Infrared Radial Artery Visualization Device 10 in accordance with an embodiment of the present invention is adapted to assist TCM practitioners to locate and identify the position of the cun, guan, and chi acupoints as shown in FIG. 3. This wrist area for palpating pulse is divided into three sections referred to as the three acupoints: cun (closest to the fingertips), guan (around the styloid process of the radius), and chi (closest to the heart) in a region approximately 3-5 cm in length as shown in FIG. 3. Each acupoint can be further divided into three depth levels (superficial, middle, and deep). In the palpating pulse process, the wrist is preferably placed at the level of the patient's heart to avoid pressure variations that may distort the pulse wave.

TCM pulse palpation at the acupoints is known by TCM practitioners to determine the health of a patient. Many problems with the patient's body or health may impact the cardiac rhythm by affecting the blood flow volume and pressure in arteries. The changes may be manifested in the pulse patterns observable at the cun, guan, and chi acupoints of the left and right wrist depending on the organs or the problems of the patient's body.

These embodiments may be advantageous as it would help the practitioner and the patient to correctly identify the location of the radial artery and the cun, guan and chi points. As the cun, guan and chi acupoints are unique for use in clinical practice for palpating pulse which happens to coincide with the radial artery, it is essential for TCM practitioners to correctly identify the location of the radial artery and in turn for the cun, guan and chi acupoints for examination and pulse taking.

The radial artery is covered by a thin layer of collagen fibres, fat, and keratinised cells which form the dermal and epidermal. The radial artery forms two distinct arterial branches. The first is the dorsal carpal branch which meets the ulna dorsal carpal branch and forms the dorsal carpal network that eventually feeds to the middle, ring, and little fingers. The second branch is the first dorsal metacarpal artery which supplies blood to the thumb and index finger.

Anatomically, the radial artery commences at the bifurcation of the brachial artery around the elbow joint running along the lateral portion of the forearm towards the wrists as shown in FIGS. 3, 4 and 5, and into the fingers of the hand. Of particular significance for pulse palpation is the radial artery at the wrist region as shown in FIG. 3 which is approximately 3-5 cm in length or around the width of three fingers, namely, the index finger, middle finger, and annular. The radial artery at this wrist region is supported by the styloid process of the radius and adjacent tendons such as the brachioradialis. Traditionally, TMC practitioners are used to locating the guan acupoint by laying the middle finger on the wrist of the patient near the styloid process.

The radial artery on the wrists is covered by a thin layer of tissue comprising collagen fibers, adipose tissue, and keratinized cells, constituting the dermal and epidermal layers. The radial artery passes through the wrist and divides into two arterial branches: the first dorsal metacarpal artery, running into the thumb and index finger; and the dorsal carpal branch which converges with the ulna dorsal carpal branch running into the middle finger, annular finger, and the little finger. When exposed to infrared light, particularly in the near-infrared light spectrum, the region of the radial artery is observed by the infrared light sensor as a darker region because the absorption of hemoglobin is higher than in other tissues.

The pulse patterns manifested at the cun, guan, and chi acupoints on the left and right wrist of a patient reflect valuable internal physiological about the entire body. The accuracy of palpation depends on the identification of the acupoints 300, which are on the radial artery in the wrist region 302, and the CMP's experiential perception as shown in FIGS. 3, 4, and 5.

Through the Infrared Radial Artery Visualization device 10 in accordance with an embodiment of the present invention as illustrated in FIG. 1, Chinese Medicine Practitioners (CMPs) may diagnose patients remotely and accurately by using a robotic device and provide appropriate TCM healthcare advice and treatment plans, saving patients time and transportation costs while improving the quality of TCM services.

The aim of an example of the Infrared Radial Artery Visualization device 10 of the present invention is to provide a non-contact optical device to assist TCM practitioners in identifying and locating the radial artery and in turn the cun, guan, and chi acupoints on the wrist. When in use, the patient will lay the wrist on platform 18 of the Infrared Radial Artery Visualization device 10. The infrared light is emitted from the infrared light source 22 and projected under the wrist of a patient. The infrared light penetrates through the wrist and is captured by the infrared sensor 12.

In one embodiment, the Infrared Radial Artery Visualization device 10 comprises a communication interface to forward the signals captured by the infrared sensor to a processor to process and display the images of the wrist. In one embodiment, the communication interface is data bus communicating with an internal processing circuit within the Infrared Radial Artery Visualization Device 10. The Radial Artery Visualization Device 10 may also comprise a display to show the resulting images in real-time.

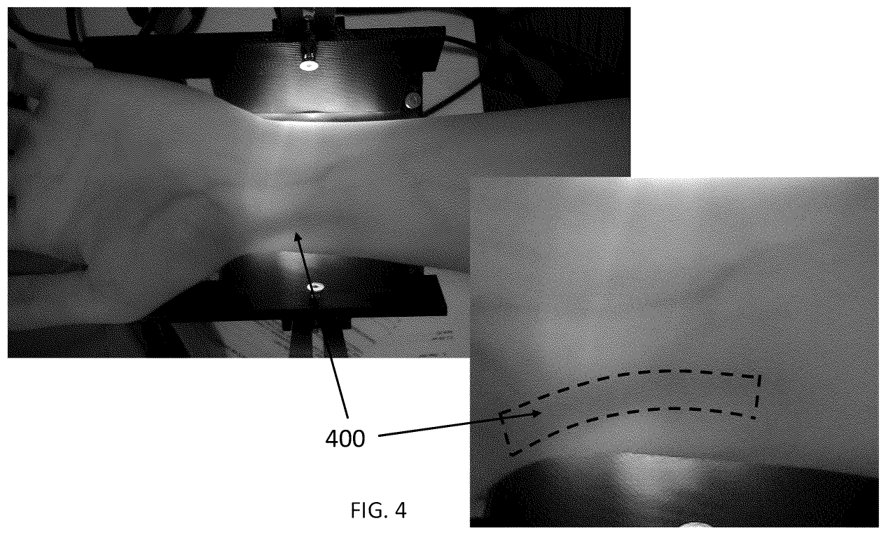

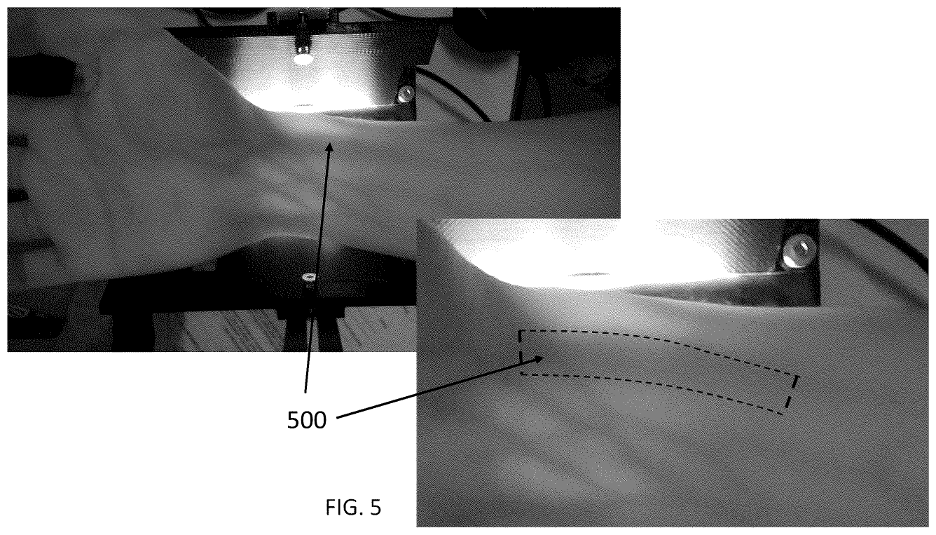

An example of the displayed images is shown in FIG. 4 and FIG. 5. The Radial Artery Visualization Device 10 is adapted to generate the images as shown in FIG. 4 and FIG. 5 to indicate a region of the radial artery that is suitable for palpation. The region of the radial artery is determined by a darker region (in dotted box, 400 and 500) as the absorption of hemoglobin is higher than in other tissues and thus producing a darker region.

Previously, some systems use a thresholding technique whereby the software converts all images taken into binary images and the variation of centroid position (amplitude variation) can be achieved. The position of the cun, guan, and chi pulses may be automatically located at points where the maximum amplitude variation is observed. Taking the time domain waveform for cun, guan, and chi pulses into fast Fourier transform (FFT) the frequency domain data is returned. This falls foul into the assumption that cun, guan and chi will have maximum amplitude variation. Also, the image processing of the prior art system is time-consuming and data-intensive.

The Infrared Radial Artery Visualization device 10, as introduced in this present invention, employs a light penetration technique to leverage the distinct absorption rates exhibited by various tissues and blood vessels. The Chinese Medicine Practitioners (CMPs) may remotely capture pulse waveforms through the utilization of the Infrared Radial Artery Visualization Device 10. This methodology proves particularly well-suited for modern/future TCM tele-practice consultation.

Embodiments of this device 10 of the present invention may be specifically designed to facilitate palpation during tele-practice and serves as a valuable tool for Chinese medicine practitioners. The Infrared Radial Artery Visualization device 10 offers the capability to locate and identify the wrist region suitable for palpation, enabling robotic devices on the subject's side (patient's side) to collect pertinent pulse information.

In one embodiment, an Infrared Radial Artery Visualization Device 10 comprises visible lighting source engaged to the frame 14 or platform 18. The visible lighting source is adapted to illuminate the surface of the wrist on the platform 18. The Infrared Radial Artery Visualization Device 10 may also comprise a camera for capturing visual light reflected from the wrist. The camera may be a 2D camera or 3D stereo camera. The image captured from the camera may be superimposed with the infrared images captured by the infrared sensor 12 for diagnosis or location of acupoints.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge unless otherwise indicated.

What is claimed is:

1. An infrared radial artery visualization device comprising
an infrared light source;
an infrared light sensor positioned opposite the infrared light source; and
a wrist support adapted to hold a wrist between the infrared light source and the sensor
wherein the sensor is adapted to capture infrared light emitted from the infrared light source and penetrated through the wrist to generate infrared wrist image or video.

2. An infrared radial artery visualization device according to claim 1, wherein the infrared light source is adapted to generate light with a wavelength from around 750 nm to 1000 μm (400 THz to 300 GHz).

3. An Infrared radial artery visualization device according to claim 1, wherein the infrared light source is adapted to emit infrared light in the near-infrared region of the electromagnetic spectrum (from 780 nm to 2500 nm).

4. An Infrared radial artery visualization device according to claim 3, wherein the infrared light source comprises one or more light-emitting diodes (LED).

5. An Infrared radial artery visualization device according to claim 4, wherein the LEDs may be arranged in an array or a panel format.

6. An Infrared radial artery visualization device according to claim 5, wherein the infrared light source comprises a circuit for modulating the cycle, pulse width, intensity, and wavelength of the infrared light emission.

7. An Infrared radial artery visualization device according to claim 6, wherein the infrared light source may comprise a temperate regulation system for regulating the temperature of the infrared light source.

8. An infrared radial artery visualization device according to claim 7, wherein the temperature regulation system may comprise one or more vents and/or fans.

9. An infrared radial artery visualization device according to claim 8, wherein the temperature regulation system may comprise a temperature sensor.

10. An infrared radial artery visualization device according to claim 9, wherein the light source is secured in an enclosure with an opening.

11. An infrared radial artery visualization device according to claim 10, wherein the opening comprises a transparent plate or a semi-opaque shading to diffuse the infrared light.

12. An infrared radial artery visualization device according to claim 11, wherein the enclosure comprises a prism, a reflector, and/or lens to direct or focus the infrared light.

13. An infrared radial artery visualization device according to claim 12, wherein the infrared light sensor is connected to a frame.

14. An infrared radial artery visualization device according to claim 13, wherein the frame forms an arch with a substantially rectangular shape.

15. An infrared radial artery visualization device according to claim 13, wherein the infrared light sensor may be connected to the frame at a hinge such that the infrared light sensor may be pivotable and/or rotatable relative to the frame.

16. An infrared radial artery visualization device according to claim 1, wherein the platform comprises fringes to assist in positing the wrist over the infrared light sensor.

17. An infrared radial artery visualization device according to claim 1, wherein the infrared sensor comprises one or more Charge Coupled Device (CCD) and Complementary Metal-Oxide-Semiconductor (CMOS) sensors.

18. An infrared radial artery visualization device according to claim 1, wherein the infrared sensor is adapted to communicate with a processor for processing image signals through one or more communication interfaces.

19. An infrared radial artery visualization device according to claim 1, wherein a diffusion lens is configured on top of the infrared light source.

20. An infrared radial artery visualization device according to claim 1, further comprising a data communication module to transmit the infrared wrist image or video for presenting on two or more display interfaces.
